(19) United States
(12) Patent Application Publication
(10) Pub. No.: US 2024/0002767 A1
(43) Pub. Date: Jan. 4, 2024

(54) Methods for Culturing Antibody Secreting Cells

(71) Applicant: Shayne Guiliano, Hillsborough, NC (US)

(72) Inventor: Shayne Guiliano, Hillsborough, NC (US)

(21) Appl. No.: 18/347,157

(22) Filed: Jul. 5, 2023

Related U.S. Application Data
(63) Continuation of application No. PCT/US2022/011446, filed on Jan. 6, 2022.
(60) Provisional application No. 63/199,560, filed on Jan. 8, 2021.

(51) Int. Cl. C12M 1/12; C12M 1/00; C12N 5/0781

(52) U.S. Cl. CPC C12M 25/10; C12M 23/20; C12N 5/0635; C12N 2502/095; C12N 2511/00

(57) ABSTRACT

The invention relates to methods for culturing antibody secreting cells, and related methods, constructs and compositions.

### RELATED APPLICATIONS

This application claims the benefit of priority under 35 U.S.C. § 119(e) to PCT Application No. PCT/US2022/011446 Jan. 6, 2022, which claims the benefit of priority to U.S. Provisional Application No. 63/199,560, filed Jan. 8, 2021, the entire contents of which is incorporated herein by reference in its entirety.

### FIELD OF THE INVENTION

This invention relates to methods for culturing antibody secreting cells for antibody production using a hollow fiber bioreactor, and related compositions and methods.

### SUMMARY OF THE INVENTION

The present invention is based, in part, on the discovery that antibody secreting cells can be successfully cultured on a cell monolayer using a hollow fiber bioreactor system. Cells cultured in accordance with the methods described here display a surprising longevity compared to other ex vivo cultures systems. Thus, in one aspect the invention relates to a bioreactor construct comprising antibody secreting cells and a cell monolayer in contact with a surface of a plurality of hollow capillary tubes arranged in a parallel array within a tubular cartridge defining an intracapillary (IC) space and an extracapillary (EC) space of the bioreactor, each hollow capillary tube constructed of a semi-permeable membrane defining an internal surface adjacent to the IC space and an external surface adjacent to the EC space, wherein the cell monolayer is in contact with the internal or external surface of the plurality of capillary tubes.

In embodiments, the internal or external surface of the plurality of capillary tubes is coated with one or more extracellular matrix components and the cell monolayer is in contact with the coated surface. In embodiments, the one or more extracellular matrix components is selected from the group consisting of collagen IV, laminin, entactin, tenascin, and fibronectin. In embodiments, a surface of each hollow capillary tube is coated with a mixture of collagen IV and laminin I. In a further embodiment, the internal or external surface of the plurality of capillary tubes is coated with one or more of a natural polymer, a biocompatible synthetic polymer, a synthetic peptide, or a composite derived from any combination thereof. In embodiments, the natural polymer is selected from the group consisting of agarose, alginate, cellulose, chitosan, gelatin, and mixtures thereof. In embodiments, the biocompatible synthetic polymer is selected from the group consisting of acrylate polymers, polyethylene co-vinyl acetate, polyethylene glycol, polysulfone, polyvinyl alcohol, polyvinylidene fluoride, sodium polyacrylate, mixtures thereof.

In embodiments, the semi-permeable membrane is fabricated from polyvinylidene difluoride (PVDF) or polysulfone. In embodiments, the semi-permeable membrane has a molecular weight cut-off (MWCO) between 5-80 kilodaltons (kDa).

In embodiments, the cell monolayer comprises primary cells or immortalized cells. In embodiments, the cell monolayer comprises mammalian cells. In embodiments, the mammalian cells are human, bovine, goat, sheep, canine, porcine, rodent, or non-human primate cells. In embodiments, the mammalian cells are human cells.

In embodiments, the cell monolayer comprises epithelial cells, fibroblast cells, or a combination thereof. In embodiments, the cell monolayer comprises mammary epithelial cells and optionally, fibroblast cells.

In embodiments, the cell monolayer comprises cells genetically engineered to express one or more proteins selected from the group consisting of CD40, polymeric immunoglobulin receptor (pIgR), and immunoglobulin J-chain.

In embodiments, the antibody secreting cells are primary B lymphocytes. In embodiments, the primary B lymphocytes are obtained from a peripheral blood mononuclear cell (PBMC) fraction, mammoplasty tissue, or mammalian milk. In embodiments, the antibody secreting cells are immortalized cells selected from the group consisting of lymphoblasts, B lymphocytes, and hybridomas. In embodiments, the antibody secreting cells are genetically engineered to express BCL-6/BCL-XL.

In embodiments, the antibody secreting cells are human cells and the cell culture medium comprises one or more B cell activating molecules selected from the group consisting of anti-CD40 antibody, anti-IgM antibody, IL-4, IL-2, and IL-10.

In embodiments, the bioreactor further comprises T helper cells.

In another aspect, the invention relates to methods for culturing antibody secreting cells, the method comprising seeding a bioreactor construct with antibody secreting cells and a second type of cell selected from epithelial cells, fibroblast cells, or a combination thereof; wherein the bioreactor construct comprises a surface of a plurality of hollow capillary tubes arranged in a parallel array within a tubular cartridge defining an intracapillary (IC) space and an extracapillary (EC) space of the bioreactor, each hollow capillary tube constructed of a semi-permeable membrane defining an internal surface adjacent to the IC space and an external surface adjacent to the EC space; wherein the second cell type forms a monolayer in contact with the internal or external surface of the plurality of capillary tubes; and wherein a cell culture medium fills the IC space or the EC space, or both.

In accordance with the methods described here, the bioreactor construct, cell monolayer, and antibody secreting cells are as described herein.

In embodiments, the methods further comprise a step of preparing the bioreactor prior to seeding the cells, wherein preparing the bioreactor comprises coating an internal or external surface of the plurality of capillary tubes with one or more extracellular matrix components followed by seeding the cells into the bioreactor such that the seeded cells are in contact with the coated surface. In embodiments, the one or more extracellular matrix components is selected from the group consisting of collagen IV, laminin, entactin, tenascin, and fibronectin. In embodiments, the one or more extracellular matrix components comprises a mixture of collagen IV and laminin I.

In a further aspect, the invention relates to immunoglobulin molecules produced by antibody secreting cells cultured according to the methods described herein. In embodiments, the immunoglobulin molecule is a secretory IgA, secretory IgM, or IgG, or a mixture of any of the foregoing, for example a polyclonal mixture. In embodiments, the IgG may be selected from any subtype, for example the IgG subtype may be selected from the group consisting of IgA1, IgA2, IgG1, IgG2, IgG3, IgG4, and allotypes; or a polyclonal mixture thereof.

In a further aspect, the invention relates to compositions comprising an immunoglobulin molecule produced in the bioreactor construct in accordance with the methods described here. In embodiments, the composition comprises a secretory IgA. In embodiments, the composition comprises milk or a milk product. In embodiments, the milk is human, bovine, goat, or sheep milk.

These and other aspects of the invention are set forth in more detail in the description of the invention below.

### BRIEF DESCRIPTION OF THE DRAWINGS

FIG. 1 shows an electron micrograph of secretory IgA dimers isolated from the supernatant of cells cultured as described in Example 1.

FIG. 2 shows increasing production of immunoglobulin molecules over time as produced in culture with mammary epithelial cells as described in Example 1.

FIG. 3A-C shows tandem mass spectrophotography histograms of immunoglobulin and immunoglobulin receptor peptides identified in the supernatant of cells cultured as described in Example 1. A, Immunoglobulin gamma-1 heavy chain (304-319); B, Immunoglobulin heavy constant alpha 1 (283-299); C, Polymeric immunoglobulin receptor (623-638).

FIG. 4. Shows a representative chromatograph of a sample of the supernatant of cells cultured as described in Example 1. Light grey line represents the full spectrum, which is overlaid with the spectra of individual proteins isolated from the sample, shown as black lines. Arrows indicate immunoglobulin and immunoglobulin receptor peaks.

### DETAILED DESCRIPTION OF THE INVENTION

The present invention relates to methods for culturing antibody secreting cells with a second type of cell forming a monolayer in a hollow fiber bioreactor. In a further aspect, the invention relates to a bioreactor construct comprising antibody secreting cells and a cell monolayer on a plurality of hollow capillary tubes arranged in a parallel array within a tubular cartridge defining an intracapillary (IC) space and an extracapillary (EC) space of the bioreactor. In a further aspect, the invention relates to immunoglobulin molecules produced by antibody secreting cells cultured in accordance with the methods described here, and related compositions.

In accordance with the present invention, antibody secreting cells are co-cultured with at least one additional cell type which forms a cell monolayer. The cell monolayer preferably comprises mammalian cells, e.g., human, bovine, goat, sheep, canine, porcine, rodent, or non-human primate cells. In one embodiment the cells are human cells. In embodiments, the cell monolayer comprises fibroblast or epithelial cells, or a combination of fibroblast and epithelial cells.

The cells of the monolayer may be primary cells or immortalized cells.

In embodiments, the cell monolayer comprises cells genetically engineered to express one or more proteins to enhance antibody production by the antibody secreting cells. In embodiments, the one or more proteins is selected from the group consisting of cluster of differentiation 40 (CD40), polymeric immunoglobulin receptor (pIgR), and immunoglobulin J-chain. Methods for genetic engineering known in the art can be used to introduce the genes encoding these proteins.

Suitable methods for genetic engineering include technologies utilizing nucleases for genome editing, such as zinc finger nucleases, transcription activator-like effector nuclease, and CRISPR/Cas9. See for example Koch et al., Nat Protoc. 2018 June; 13(6): 1465-1487; and a review of CRISPR/Cas systems in Nat Biotechnol 2014 Apr.; 32(4):347-55. More traditional recombinant technologies which utilize bacterial or viral vectors may also be used and are described, for example, in Green and Sambrook, Molecular Cloning Cold Spring Harbor Laboratory Press 2012. See e.g., chapters 3-5, and 15, 16.

In embodiments, the cell monolayer comprises mammary epithelial cells. In embodiments, the mammary epithelial cells are milk producing cells. In embodiments, the milk producing mammary epithelial cells are primary cells, or primary immortalized cells.

The antibody secreting cells may also be primary or immortalized cells. In embodiments, the antibody secreting cells are primary B lymphocytes. Primary B lymphocytes may be obtained according to methods known in the art, for example from a peripheral blood mononuclear cell (PBMC) fraction, mammoplasty tissue, or mammalian milk.

In embodiments, the antibody secreting cells are immortalized cells selected from the group consisting of lymphoblasts, B lymphocytes, and hybridomas. In embodiments, the methods described here encompass immortalizing a primary cell.

Methods for immortalizing cells are known in the art. For example, cells may be immortalized by introduction of a virus or viral gene, e.g., using Epstein Barr virus, SV40 virus, human papillomavirus (HPV) sequences, human T-lymphotropic virus type 1 (HTLV-1); by genetically engineering the cells to express certain proteins, such as BCL-6/BCL-XL, TERT, or telomerase; or by fusing the cells with an immortal cell line to form a hybridoma. Combinations of the foregoing techniques may also be used. In embodiments, the cells are immortalized through genetic engineering to express BCL-6/BCL-XL.

In embodiments, the antibody secreting cells are genetically engineered to express one or more molecules selected from the group consisting of a F(ab) fragment, a polymeric immunoglobulin receptor (pIgR), and an immunoglobulin J-chain. In embodiments, the antibody secreting cells are genetically engineered to delete the gene encoding activation-induced cytidine deaminase (AID), the AICDA gene.

In embodiments, the antibody secreting cells are primary B lymphocytes, preferably isolated from milk, blood, or mammoplasty tissue, preferably human but can be from other mammalian species e.g., bovine, goat, sheep, canine, porcine, rodent, or non-human primate. In embodiments the primary B lymphocytes are genetically engineered to express one or more molecules including a F(ab) fragment with desired affinity, a polymeric immunoglobulin receptor (pIgR), and an immunoglobulin J-chain; or to delete one or more genes, such as the AICDA gene. In a further embodiment, the genetically engineered primary B lymphocytes are immortalized. In embodiments, the primary cells are immortalized using hybridoma technology or by further genetically engineering the cells to express a suitable protein, such as BCL-6/BCL-XL, TERT, or telomerase.

Methods for genetic engineering of primary B lymphocytes are described, for example, in Johnson et al., Sci Rep 8, 12144 (2018).

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

As used herein, the term “about,” as used herein when referring to a measurable value such as an amount of a compound or agent of this invention, dose, time, temperature, and the like, is meant to encompass variations of less than ±1-5% of the specified amount.

As used herein, the transitional phrase “consisting essentially of” is to be interpreted as encompassing the recited materials or steps and those that do not materially affect the basic and novel characteristic(s) of the claimed invention. This term is not equivalent to the open-ended “comprising.”

As used herein, the term “protein” encompasses peptides, polypeptides and proteins, unless indicated otherwise.

As used herein, by “isolate” (or grammatical equivalents, e.g., “extract”) a product, it is meant that the product is at least partially separated from at least some of the other components in the starting material.

As used herein, the term “genetically modified or engineered” encompasses materials produced by recombinant technology.

### Bioreactor Constructs

The present invention relates to bioreactor constructs comprising antibody secreting cells and a second type of cell forming a monolayer, as discussed above. The bioreactor construct consists of at least one tubular cartridge housing a plurality of hollow capillary tubes arranged in a parallel array within the cartridge and defining an intracapillary (IC) space and an extracapillary (EC) space of the bioreactor construct. Each hollow capillary tube is constructed of a semi-permeable membrane defining an internal surface adjacent to the IC space and an external surface adjacent to the EC space. In embodiments, the capillaries are fabricated from PVDF, polysulfone, or other biologically suitable materials.

In embodiments, before seeding cells into the bioreactor, the bioreactor capillaries are first coated with an extracellular matrix (ECM) material. The ECM may comprise one or more ECM components selected from the group consisting of chondroitin sulfate, collagen IV, elastin, fibronectin, heparan sulfate, hyaluronic acid, keratin sulfate, laminin, nidogen-1 (NID-1, also known as entactin) and tenascin. In embodiments, the ECM comprises or consists of a 1:1 mixture of collagen IV and laminin I in PBS.

In embodiments, before seeding cells into the bioreactor, the bioreactor capillaries are first coated with one or more of a natural polymer, a biocompatible synthetic polymer, a synthetic peptide, or a composite derived from any combination thereof. Suitable natural polymers include agarose, alginate, cellulose, chitosan, and gelatin. Suitable biocompatible synthetic polymers include acrylate polymers, polyethylene co-vinyl acetate, polyethylene glycol, polysulfone, polyvinyl alcohol, polyvinylidene fluoride, sodium polyacrylate, and mixtures thereof.

### Methods

The present invention provides methods for co-culturing antibody secreting cells with a second type of cell that forms a monolayer on a surface of the capillary tubes within a hollow fiber bioreactor. Thus, the methods employ a hollow fiber bioreactor construct, as described above.

Standard conditions suitable for the culture of mammalian cells may be used in co-culturing the antibody secreting cells with a second type of cell, as described herein. For example, a controlled environment within the bioreactor construct suitable for culture of mammalian cells may comprise a temperature in the range of 35-39° C., preferably about 37° C.; in an atmosphere of 4-6% CO₂, preferably about 5% CO₂. The cells are cultured in a minimum or basal” cell growth medium which includes a carbon source, a buffering system to maintain a neutral pH, one or more essential amino acids, one or more vitamins and/or cofactors, and one or more inorganic salts.

Any suitable buffering system may be used for the basal growth medium. An exemplary system is sodium bicarbonate and/or 4-(2-hydroxyethyl)-1-piperazineethanesulfonic acid (HEPES).

In embodiments, the basal culture medium comprises one or more essential amino acids in an amount from about 0.5-5 mM. In some embodiments, the one or more essential amino acids is selected from arginine and cysteine, or both. In embodiments, the basal culture medium comprises one or more vitamins and/or cofactors in an amount from about 0.01-50 μM. In some embodiments, one or more vitamins and/or cofactors is selected from thiamine and riboflavin, or both. In embodiments, the basal culture medium comprises one or more inorganic salts in an amount from about 100-150 mg/L. In some embodiments, the one or more inorganic salts is selected from calcium and magnesium, or both.

Suitable basal cell growth mediums are known in the art and include, for example, Ames Medium, Basal Media Eagle, Minimum Essential Medium Eagle (MEM), Dulbecco's Modified Eagle's Media (DMEM), Iscove's Modified Dulbecco's Medium (IMDM). In some embodiments, the basal cell growth medium is DMEM supplemented with a chemically defined medium for high density cell culture, such as FiberCell Systems CDM-HD.

In embodiments, the basal cell growth medium is supplemented with one or more growth factors, hormones, or B cell activating molecules. In embodiments, the basal cell growth medium is supplemented with one or more of epidermal growth factor, prolactin, and a B cell activating molecule selected from the group consisting of anti-CD40 antibody, anti-IgM antibody, IL-4, IL-2, and IL-10, and combinations thereof.

In embodiments, both the antibody secreting cells and the second cell type are seeded together into the bioreactor. In embodiments, the antibody secreting cells are seeded after the second cell type. In embodiments, the methods described here may further comprise seeding additional antibody secreting cells into the bioreactor at one or more times following the initial seeding.

In embodiments, cells are seeded into the extracapillary (EC) space, and a basal medium is pumped through the capillaries, into the intracapillary space (IC). In an alternative embodiment, the cells are seeded into the IC space and the EC space contains the basal medium.

Prior to seeding the cells in the bioreactor, it is generally advantageous to culture the cells in order to obtain an expanded population of cells in a phase of exponential growth. Accordingly, the methods described here may further comprise a step of culturing the antibody secreting cells and/or the second cell type prior to seeding the cells into the bioreactor in order to obtain an expanded population of cells, preferably in a phase of exponential growth at the time of seeding. Cells are seeded into the bioreactor using standard cell culture protocols.

The second cell type may be any type of cell capable of forming a monolayer on a surface of the capillaries, for example fibroblast or epithelial cells. The monolayer forming cells may be derived from any organ, including for example, mammary, lung, skin, kidney, colon, etc.

In embodiments, the second cell type comprises mammary epithelial cells and optionally, fibroblast cells. In embodiments, the methods further comprise stimulating milk production in the mammary epithelial cells, which may be primary cells or immortalized cells, including immortalized primary cells. In an embodiment, stimulating milk production comprises contacting a confluent monolayer of the mammary epithelial cells with a first amount of prolactin and culturing the cells for a period of time; followed by contacting the cells with a second amount of prolactin and culturing the cells for a second period of time. In embodiments, the first amount of prolactin is from about 80-150 ng/ml, preferably about 100 ng/ml; and the second amount of prolactin is from about 150-250 ng/ml, preferably about 200 ng/ml, supplemented in the basal culture medium. In embodiments, the first period of time is from about 3-15 days; and the second period of time is from about 5-20 days.

The formation of a cell monolayer in the bioreactor can be monitored, for example, by determining glucose utilization over a period of days following seeding the cells into the bioreactor. Glucose utilization is an indicator of cellular metabolism. During exponential growth, glucose utilization increases sharply, then slows and drops to a lower steady state when the cells reach confluence. Confluent cells will form a barrier dividing the intracapillary (IC) and extracapillary (ECS) spaces. The integrity of the monolayer may be determined, for example, using a transepithelial electrical resistance (TEER) assay. TEER measures a voltage difference between the fluids in the two compartments. Where barrier integrity is lost, and two fluids mix, the voltage difference drops to zero. Conversely, a voltage difference indicates that the barrier is intact.

In accordance with the methods described here, in some embodiments the basal cell culture medium of the bioreactor is supplemented with one or more B cell activating molecules selected from the group consisting of anti-CD40 antibody, anti-IgM antibody, IL-4, IL-2, and IL-10. These and similar agents are commercially available, for example from Sigma Aldrich, Irvine Scientific, and Schering Plough. Suitable methods for the in vitro activation of antibody secreting B cells are described, for example, in Lefevre et al. J Immunol 1999; 163:1119-1122.

In an embodiment, the methods described here can be used to produce fully human antibodies, either with or without genetic modifications. In addition, the methods described here provide improvements in antibody production attainable due to the ability to continuous monitor the amount and types of antibodies present by sampling of the bioreactor supernatant through the port. Through such continuous monitoring, it is possible to control both the overall production of antibodies as well as the ratios of monoclonal and polyclonal mixtures. For example, additional antibody secreting cells may be added to maintain or achieve a target formulation containing a specific ratio of neutralizing antibodies for therapeutic use.

In embodiments, the antibody secreting cells are human cells secreting neutralizing antibodies against an antigenic molecule of a pathogen, such as a virus or bacteria. In embodiments, the antigenic molecule is the spike protein of SARS-CoV-2. In embodiments, the neutralizing antibodies are monoclonal antibodies. In embodiments, the neutralizing antibodies are polyclonal antibodies. Methods for producing human cells secreting neutralizing antibodies against a target antigenic molecule (also referred to as an ‘immunogen’) are known in the art and can be used to produce suitable antibody secreting cells for use in the methods described here. For example, as described in Boonyaratanakornit and Taylor, Front. Immunol 2019.

In embodiments, the present methods further provide a fully human system for the production of antibodies that is free of endotoxin and other impurities, such that the antibodies produced according to the methods described here do not require the post-processing steps typically needed to remove such contaminants prior to use in humans. This is achieved in part by ensuring that all components of the bioreactor system are “food grade” in accordance with the definition of regulatory authorities, such as the US Food and Drug Administration. Thus, in embodiments, the basal culture medium, ECM materials, etc. of the bioreactor system are comprised of food grade ingredients.

In embodiments, the cell monolayer is a milk producing mammary epithelial cell monolayer and the components of the bioreactor system are food grade materials, including without limitation the basal culture medium, media supplements, and the ECM materials. In accordance with this embodiment, the invention further provides a food grade biosynthetic milk product containing antibodies. In embodiments, the antibody containing biosynthetic milk product is unpasteurized. In embodiments, the invention further provides a composition comprising the antibody containing biosynthetic milk product.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant molecules, manipulation of nucleic acid sequences, production of transformed cells, the construction of viral vector constructs, and transiently and stably transfected packaging cells. Such techniques are known to those skilled in the art. See, e.g., Sambrook et al., Molecular Cloning: A Laboratory Manual 2nd Ed. (Cold Spring Harbor, N Y, 1989); F. M. Ausubel et al. Current Protocols In Molecular Biology (Green Publishing Associates, Inc. and John Wiley & Sons, Inc., New York).

All publications, patent applications, patents, nucleotide sequences, amino acid sequences and other references mentioned herein are incorporated by reference in their entireties for the teachings relevant to the sentence and/or paragraph in which the reference is presented.

Having described the present invention, the same will be explained in greater detail in the following examples, which are included herein for illustration purposes only, and which are not intended to be limiting to the invention.

### EXAMPLES

### Example 1

The following describes the cultivation of primary antibody secreting B cells in a bioreactor seeded with primary human mammary epithelial cells (HUMECs). In this example, the antibody secreting B cells were not separately seeded into the bioreactor. As discussed below, biochemical analyses of the supernatant of the bioreactor identified numerous immunoglobulin molecules, including secretory IgA dimers, demonstrating the presence of antibody secreting B cells.

### Preparation of Hollow Fiber Bioreactor:

Prior to seeding with cells, the bioreactor cartridge was prepared by incubation with PBS for a minimum of 24 hours followed by coating the capillaries with a 1:1 mixture of collagen IV and laminin I (25 μg Laminin-111, 25 μg Collagen IV) in PBS at room temperature overnight. The collagen/laminin mixture was then exchanged with cell growth medium and incubated overnight at room temperature.

### Culture of Cells in the Bioreactor:

Primary human mammary epithelial cells (HUMECs) were obtained from the American Type Culture Collection (ATCC®) and expanded in accordance with standard protocls before seeding into the bioreactor. After seeding, cells were allowed to proliferate until confluence was reached, as determined by glucose utilization. Cells were cultured in a basal growth medium (ATCC® PCS-600-030™) until confluence. Following confluence, the basal medium was supplemented with Dulbecco's Modified Eagle's Medium (DMEM, Sigma Aldrich) containing a chemically defined medium for high density cell culture (FiberCellSystems CDM-HD, 10% by vol. in basal medium).

Milk production was stimulated with 100 ng/ml prolactin followed by increasing prolactin to 200 ng/ml.

Samples of culture supernatant were collected from the bioreactor port hole for subsequent analyses.

### Characterization of Antibody-Secreting B Cells

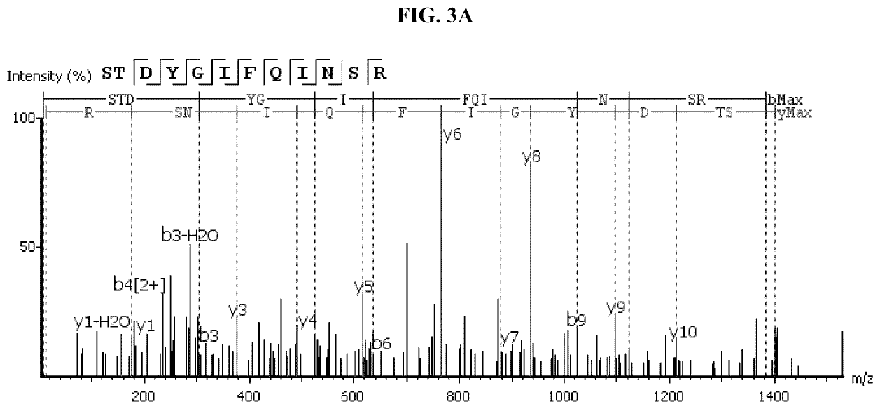

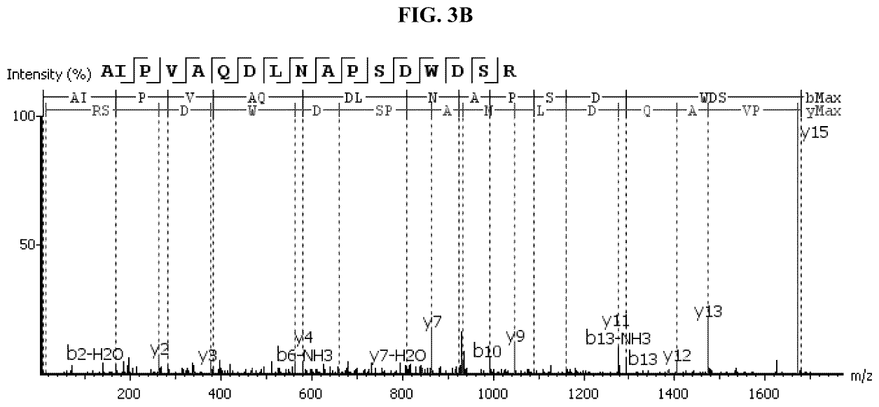

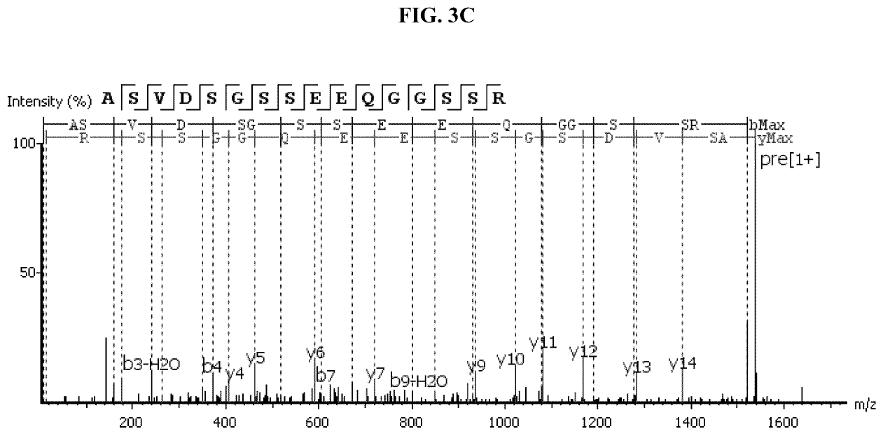

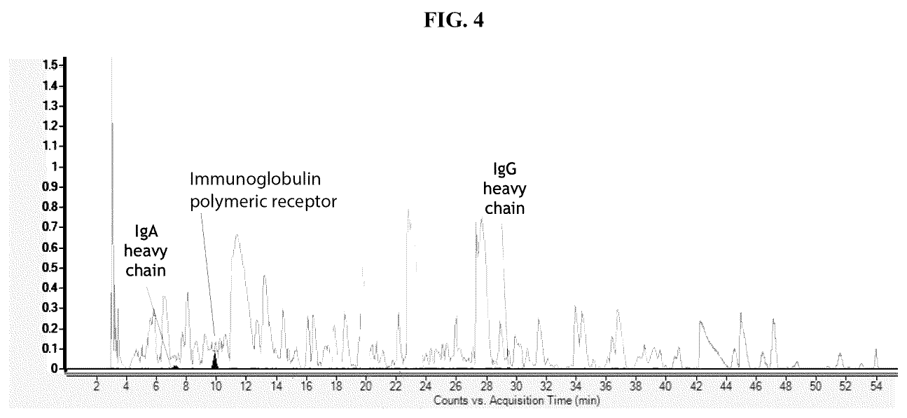

The presence of antibody secreting B cells in the bioreactor is evidenced by the detection of various immunoglobulin molecules in samples collected from the bioreactor. Immunoglobulin molecules were detected by liquid chromatography coupled with mass spectrophotometry (LC-MS) and by tandem mass spectrophotometry (MS-MS) using standard protocols. Representative MS chromatographs of proteins isolated from a sample of the supernatant of cells cultured as described here are shown in FIG. 3A-C. The figure depicts peptides representing immunoglobulin gamma-1 heavy chain (304-319), immunoglobulin heavy constant alpha 1 (283-299), and polymeric immunoglobulin receptor (623-638), respectively. A representative chromatograph showing peaks for the whole sample (light grey line) overlaid with the peaks from the three isolated proteins (black filled areas) is shown in FIG. 4. Table 1 below lists all of the immunoglobulin molecules identified in the culture supernatant.

TABLE 1
Immunoglobulin molecules identified in bioreactor supernatant

| | |
|---|---|
| P0DOX2\|IGA2_HUMAN | Immunoglobulin alpha-2 |
| | heavy chain |
| P0DOX5\|IGG1_HUMAN | Immunoglobulin gamma-1 |
| | heavy chain |
| P01877\|IGHA2_HUMAN | Immunoglobulin heavy |
| | constant alpha 2 |
| P01857\|IGHG1_HUMAN | Immunoglobulin heavy |
| | constant gamma 1 |
| P01859\|IGHG2_HUMAN | Immunoglobulin heavy |
| | constant gamma 2 |
| P01860\|IGHG3_HUMAN | Immunoglobulin heavy |
| | constant gamma 3 |
| P01861\|IGHG4_HUMAN | Immunoglobulin heavy |
| | constant gamma 4 |
| P01871\|IGHM_HUMAN | Immunoglobulin heavy |
| | constant mu |
| P01591\|IGJ_HUMAN | Immunoglobulin J chain |
| P0DOY2\|IGLC2_HUMAN | Immunoglobulin lambda |
| | constant 2 |
| P0DOY3\|IGLC3_HUMAN | Immunoglobulin lambda |
| | constant 3 |
| B9A064\|IGLL5_HUMAN | Immunoglobulin lambda-like |
| | polypeptide 5 |
| P0DOX6\|IGM_HUMAN | Immunoglobulin mu heavy |
| | chain |

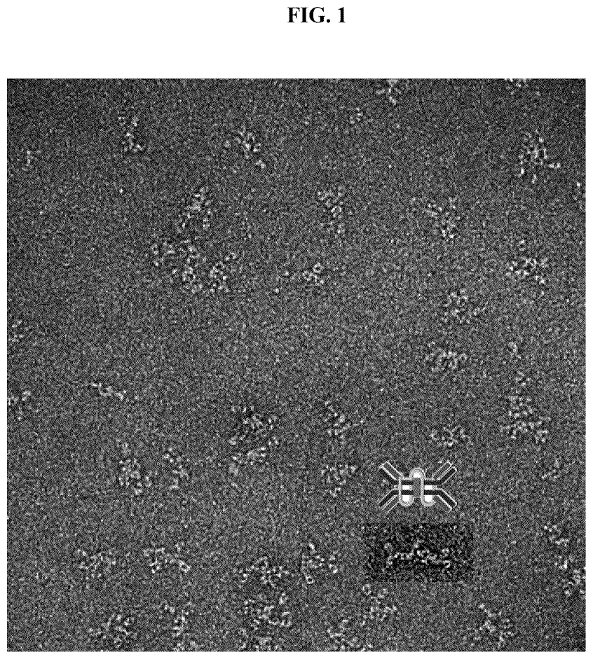

In addition, secretory IgA dimers were also identified as evidenced by the electron micrograph shown in FIG. 1.

In addition, production of secretory IgA dimers was quantified by use of a colorimetric Human Secretory IgA ELISA Kit produced by Novus Biologicals. ELISA analysis demonstrated a peak production of 250 mg of SIgA per day per liter of ECS, an unprecedented level of efficiency for biosynthesis of secretory IgA dimers. The production of 250 mg/day/L-ECS was extrapolated from producing 8 mg of sIgA over 10 days in 3 mL ECS. The invention described herein surprisingly showed the high total amount of SIgA produced, e.g., 8 mg over 10 days, in a small volume.

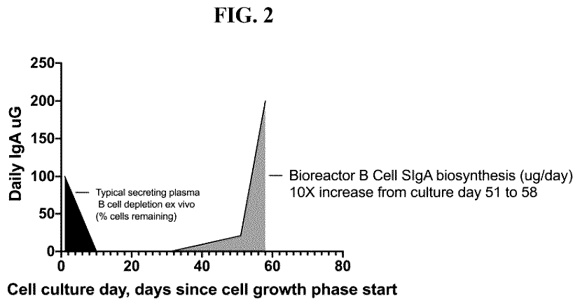

Antibody secreting plasma B cells are notoriously short lived ex vivo when isolated from blood, milk or tissue, with reported half lives of 3-5 days without molecular or genetic modification. As shown in FIG. 2, the methods described here supported a much longer-term survival of non-modified antibody secreting cells than expected. In a typical ex vivo plasma cell culture, the antibody secreting cell population would be expected to be depleted by about day 10 of culture. Instead, in the system described here, around ex vivo day 51-58, antibody production substantially increased. Without being bound by any particular theory, co-culture with epithelial monolayers as described here maximizes the survival of antibody secreting cells, as well as their production of antibody. These methods can be used to produce, for example, fully human polyclonal antibodies of any desired class or subtype at therapeutic scale, without the need to genetically modify the antibody secreting cells. Of course, targeted genetic modifications could also be used to further enhance the cells' survival, their antibody production, and antigen affinity, as described herein.

### Production of Antibodies at Commercially Feasible Scale

The methods described here provide a proof-of-concept for the production of antibodies using bioreactor cultured antibody secreting cells. The hollow fiber bioreactor is a particularly advantageous system for maximizing surface area while allowing the cells to organize into three dimensional structures. The process is readily scalable for commercial production. In the present example, a relatively small bioreactor cartridge was used to culture the epithelial monolayer. It contained about 400 square centimeter (cm²) of surface area for cell growth. Using a larger commercially available bioreactor cartridge would substantially increase the surface area and consequently the production of antibodies. For example, larger commercially available bioreactors may have a surface area of about 3 square meters (m²). The process is further scalable, for example, by packing more fibers and/or longer fibers into one or more cartridges aligned in parallel.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

What is claimed is:

1. A bioreactor construct comprising antibody secreting cells and a cell monolayer in contact with a surface of a plurality of hollow capillary tubes arranged in a parallel array within a tubular cartridge defining an intracapillary (IC) space and an extracapillary (EC) space of the bioreactor, each hollow capillary tube constructed of a semi-permeable membrane defining an internal surface adjacent to the IC space and an external surface adjacent to the EC space, wherein the cell monolayer is in contact with the internal or external surface of the plurality of capillary tubes and wherein a cell culture medium fills the IC space or the EC space, or both.

2. The bioreactor construct of claim 1, wherein the internal or external surface of the plurality of capillary tubes is coated with one or more extracellular matrix components and the cell monolayer is in contact with the coated surface.

3. The bioreactor construct of claim 2, wherein the one or more extracellular matrix components is selected from the group consisting of collagen IV, laminin, entactin, tenascin, and fibronectin.

4. The bioreactor construct of claim 3, wherein a surface of each hollow capillary tube is coated with a mixture of collagen IV and laminin I.

5. The bioreactor construct of claim 1, wherein the internal or external surface of the plurality of capillary tubes is coated with one or more of a natural polymer, a biocompatible synthetic polymer, a synthetic peptide, or a composite derived from any combination thereof.

6. The bioreactor construct of claim 5, wherein the natural polymer is selected from the group consisting of agarose, alginate, cellulose, chitosan, gelatin, and mixtures thereof.

7. The bioreactor construct of claim 5, wherein the biocompatible synthetic polymer is selected from the group consisting of acrylate polymers, polyethylene co-vinyl acetate, polyethylene glycol, polysulfone, polyvinyl alcohol, polyvinylidene fluoride, sodium polyacrylate, mixtures thereof.

8. The bioreactor construct of any one of claims 1-7, wherein the semi-permeable membrane is fabricated from polyvinylidene difluoride (PVDF) or polysulfone.

9. The bioreactor construct of claim 8, wherein the semi-permeable membrane has a molecular weight cut-off (MWCO) between 5-80 kilodaltons (kDa).

10. The bioreactor construct of any one of claims 1-9, wherein the cell monolayer comprises primary cells or immortalized cells.

11. The bioreactor construct of any one of claims 1-10, wherein the cell monolayer comprises mammalian cells.

12. The bioreactor construct of claim 11, wherein the mammalian cells are human, bovine, goat, sheep, canine, porcine, rodent, or non-human primate cells.

13. The bioreactor construct of claim 12, wherein the mammalian cells are human cells.

14. The bioreactor construct of any one of claims 1-13, wherein the cell monolayer comprises epithelial cells, fibroblast cells, or a combination thereof.

15. The bioreactor construct of claim 14, wherein the cell monolayer comprises mammary epithelial cells and optionally, fibroblast cells.

16. The bioreactor construct of any one of claims 1-15, wherein the cell monolayer comprises cells genetically engineered to express one or more proteins selected from the group consisting of CD40, polymeric immunoglobulin receptor (pIgR), and immunoglobulin J-chain.

17. The bioreactor construct of any one of claims 1-16, wherein the antibody secreting cells are primary B lymphocytes.

18. The bioreactor construct of claim 17, wherein the primary B lymphocytes are obtained from a peripheral blood mononuclear cell (PBMC) fraction, mammoplasty tissue, or mammalian milk.

19. The bioreactor construct of any one of claims 1-16, wherein the antibody secreting cells are immortalized cells selected from the group consisting of lymphoblasts, B lymphocytes, and hybridomas.

20. The bioreactor construct of claim 19, wherein the antibody secreting cells are genetically engineered to express BCL-6/BCL-XL.

21. The bioreactor construct of any one of claims 1-20, wherein the antibody secreting cells are human cells and the cell culture medium comprises one or more B cell activating molecules selected from the group consisting of anti-CD40 antibody, anti-IgM antibody, IL-4, IL-2, and IL-10.

22. The bioreactor construct of any one of claims 1-21, wherein the bioreactor further comprises T helper cells.

23. A method for culturing antibody secreting cells, the method comprising seeding a bioreactor construct with antibody secreting cells and a second type of cell selected from epithelial cells, fibroblast cells, or a combination thereof; wherein the bioreactor construct comprises a surface of a plurality of hollow capillary tubes arranged in a parallel array within a tubular cartridge defining an intracapillary (IC) space and an extracapillary (EC) space of the bioreactor, each hollow capillary tube constructed of a semi-permeable membrane defining an internal surface adjacent to the IC space and an external surface adjacent to the EC space; wherein the second cell type forms a monolayer in contact with the internal or external surface of the plurality of capillary tubes; and wherein a cell culture medium fills the IC space or the EC space, or both.

24. The method of claim 23, wherein the bioreactor construct is as described in any one of claims 2-9.

25. The method of claim 23 or 24, wherein the cell monolayer is as described in any one of claims 10-16.

26. The method of any one of claims 23-25, wherein the antibody secreting cells are as described in any one of claims 17-21.

27. The method of any one of claims 23-26, wherein the bioreactor further comprises T helper cells.

28. The method of any one of claims 23-27, further comprising a step of preparing the bioreactor prior to seeding the cells, wherein preparing the bioreactor comprises coating an internal or external surface of the plurality of capillary tubes with one or more extracellular matrix components followed by seeding the cells into the bioreactor such that the seeded cells are in contact with the coated surface.

29. The method of claim 28, wherein the one or more extracellular matrix components is selected from the group consisting of collagen IV, laminin, entactin, tenascin, and fibronectin.

30. The method of claim 29, wherein the one or more extracellular matrix components comprises a mixture of collagen IV and laminin I.

31. An immunoglobulin molecule produced by antibody secreting cells cultured according to the method of any one of claims 23-30.

32. The immunoglobulin molecule of claim 31, wherein the immunoglobulin molecule is a secretory IgA, secretory IgM, or IgG, or a mixture of any of the foregoing.

33. A composition comprising the immunoglobulin molecule of claim 31 or 32.

34. The composition of claim 33, wherein the composition comprises milk or a milk product.

35. The composition of claim 34, wherein the milk is human milk or other mammalian milk.
